(19) United States
(12) Patent Application Publication
(10) Pub. No.: US 2017/0113977 A1
(43) Pub. Date: Apr. 27, 2017

(54) UREA PARTICLES, METHODS OF MANUFACTURE, AND USES

(71) Applicant: Agra Holdings LLC, Firestone, CO (US)

(72) Inventor: Adam B. Benton, Firestone, CO (US)

(21) Appl. No.: 15/296,806

(22) Filed: Oct. 18, 2016

Related U.S. Application Data
(60) Provisional application No. 62/245,643, filed on Oct. 23, 2015.

(51) Int. Cl. C05C 9/00; C05G 3/00; A23K 20/105; A23K 20/158; A23K 20/147; C07C 275/00; A23K 40/30

(52) U.S. Cl. CPC C05C 9/00; C07C 275/00; C05C 9/005; C05G 3/0011; A23K 40/30; A23K 20/158; A23K 20/147; A23K 20/105

(57) ABSTRACT

The disclosure relates to compositions comprising urea particles. The disclosure further provides methods of manufacturing the urea particles and their uses.

### RELATED APPLICATIONS

This application claims priority under 35 U.S.C. 119 (e) to U.S. Provisional Patent Application Ser. No. 62/245,643, entitled “UREA PARTICLES, METHODS OF MANUFACTURE, AND USES”, filed Oct. 23, 2015, the disclosure of which is hereby incorporated by reference in its entirety.

Urea is a relatively inexpensive source of nitrogen for plants and animals. As a dietary source of nitrogen, urea can be used to augment the feed of certain animals such as ruminants and has been partly used as an economical replacement for animal dietary proteins.

In this sense, ruminants are known to slowly break down urea or other non-protein nitrogen sources into ammonia in the rumen. An animal's rumen contains microorganisms that help in combining ammonia with the products of carbohydrate metabolism to synthesize essential amino acids and proteins.

The granular and prill forms of conventionally available urea usually comes in sizes that are often too big for making uniform or homogeneous mixtures with other ingredients. For example, in the manufacturing of animal feed pellets, inclusion of conventional sized urea can result in feed pellets that are broken or feed pellets having urea protruding therefrom. Broken feed pellets and feed pellets having protruding urea have proven undesirable for animal feed consumption. Further, during the feed manufacturing, larger conventionally available sized urea tends to settle and concentrate at the bottom, thereby creating a less homogeneous feed mixture, again causing a less desirable feed for the animal. It has also been shown that urea, being hygroscopic, dehydrates other dietary ingredients in animal feed, particularly when the urea is broken or crushed with other ingredients, thereby making a rigid mass or new adduct compounds. Formation of adduct compounds in the feed applications can have significant ramifications for storage as the adducts tend to interact, forming undesirable feed compounds. The present invention is directed toward overcoming one or more of the problems discussed above.

Disclosed herein are various embodiments of urea in a particle size for enhanced use in plant and animal nutrition, resin production, ethanol production, yeast fermentation and cellulose paper manufacturing. Accordingly, embodiments herein provide compositions comprising these appropriately sized urea particles, and methods of manufacturing the appropriately sized urea particles. Note that the term “appropriately sized” urea particles as used herein refers to the urea particles that fit within the size parameter described in the embodiments herein.

In one embodiment, a composition comprising urea particles of appropriate size is provided. The composition as described herein may be used as an ingredient or nutrient in animal feed, plant fertilizer, resin production, ethanol production, yeast fermentation and cellulose paper manufacturing. In one aspect, appropriately sized urea particles comprise a size greater than about 15 SGN to less than about 200 SGN, e.g., greater than about 50 to less than about 200 SGN, e.g., greater than about 80 SGN to less than about 200 SGN, e.g., greater than about 100 SGN to less than about 200 SGN, e.g., greater than about 130 SGN to less than about 200 SGN, e.g., and about 135 SGN, 145 SGN, 150 SGN, 155 SGN, 160 SGN, 165 SGN, 170 SGN, 175 SGN, 180 SGN, 185 SGN, 190 SGN, 195 SGN, 196 SGN, 197 SGN, 198 SGN, or 199 SGN. In another aspect, the composition comprises urea in the form of urea particles having a size greater than about 15 SGN to less than about 200 SGN, wherein about 50% of the urea particles have the described particle size, and more typically about, e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79,%, 80%, 81%, 82%, 83%, 84%, 85%, 86%, 87%, 88%, 89%, 90%, 91%, 92%, 93%, 94%, 95%, 96%, 97%, 98%, 99% or 100% have the described particle size.

In another embodiment, the composition comprises urea particles having a size greater than 15 SGN to less than 200 SGN, e.g., greater than 50 to less than 200 SGN, e.g., greater than 80 SGN to less than 200 SGN, e.g., greater than 100 SGN to less than 200 SGN, e.g., greater than 130 SGN to less than 200 SGN, e.g., about 135 SGN, 145 SGN, 150 SGN, 155 SGN, 160 SGN, 165 SGN, 170 SGN, 175 SGN, 180 SGN, 185 SGN, 190 SGN, 195 SGN, 196 SGN, 197 SGN, 198 SGN, or 199 SGN. In another embodiment, the composition comprises urea in the form of urea particles having a size greater than 15 SGN to less than 200 SGN, wherein about 50% of the urea particles have the described particle size, and more typically about, e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79,%, 80%, 81%, 82%, 83%, 84%, 85%, 86%, 87%, 88%, 89%, 90%, 91%, 92%, 93%, 94%, 95%, 96%, 97%, 98%, 99% or 100% have the described particle size.

In another embodiment, a composition is provided consisting of urea in the form of urea particles having a size greater than about 15 SGN to less than about 200 SGN, e.g., greater than about 50 to less than about 200 SGN, e.g., greater than about 80 SGN to less than about 200 SGN, e.g., greater than about 100 SGN to less than about 200 SGN, e.g., greater than about 130 SGN to less than about 200 SGN, e.g., about 135 SGN, 145 SGN, 150 SGN, 155 SGN, 160 SGN, 165 SGN, 170 SGN, 175 SGN, 180 SGN, 185 SGN, 190 SGN, 195 SGN, 196 SGN, 197 SGN, 198 SGN, or 199 SGN. In another embodiment, provided is a composition consisting of urea in the form of urea particles having a size greater than about 15 SGN to less than about 200 SGN, wherein about 50% of the urea particles have the described particle size, and more typically about, e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79,%, 80%, 81%, 82%, 83%, 84%, 85%, 86%, 87%, 88%, 89%, 90%, 91%, 92%, 93%, 94%, 95%, 96%, 97%, 98%, 99% or 100% have the described particle size.

In another embodiment, the composition consists of urea in the form of urea particles having a size greater than 15 SGN to less than 200 SGN, e.g., greater than 50 to less than 200 SGN, e.g., greater than 80 SGN to less than 200 SGN, e.g., greater than 100 SGN to less than 200 SGN, e.g., greater than 130 SGN to less than 200 SGN, e.g., about 135 SGN, 145 SGN, 150 SGN, 155 SGN, 160 SGN, 165 SGN, 170 SGN, 175 SGN, 180 SGN, 185 SGN, 190 SGN, 195 SGN, 196 SGN, 197 SGN, 198 SGN, or 199 SGN. In another embodiment, the composition consists of urea in the form of urea particles having a size greater than 15 SGN to less than 200 SGN, wherein about 50% of the urea particles have the described particle size, and more typically about, e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79,%, 80%, 81%, 82%, 83%, 84%, 85%, 86%, 87%, 88%, 89%, 90%, 91%, 92%, 93%, 94%, 95%, 96%, 97%, 98%, 99% or 100% have the described particle size.

In another embodiment, provided is a composition consisting essentially of urea in the form of urea particles having a size greater than about 15 SGN to less than about 200 SGN, e.g., greater than about 50 to less than about 200 SGN, e.g., greater than about 80 SGN to less than about 200 SGN, e.g., greater than about 100 SGN to less than about 200 SGN, e.g., greater than about 130 SGN to less than about 200 SGN, e.g., about 135 SGN, 145 SGN, 150 SGN, 155 SGN, 160 SGN, 165 SGN, 170 SGN, 175 SGN, 180 SGN, 185 SGN, 190 SGN, 195 SGN, 196 SGN, 197 SGN, 198 SGN, or 199 SGN. In another embodiment, provided is a composition consisting essentially of urea in the form of urea particles having a size greater than about 15 SGN to less than about 200 SGN, wherein about 50% of the urea particles have the described particle size, and more typically about, e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79,%, 80%, 81%, 82%, 83%, 84%, 85%, 86%, 87%, 88%, 89%, 90%, 91%, 92%, 93%, 94%, 95%, 96%, 97%, 98%, 99% or 100% have the described particle size.

In another embodiment, provided is a composition consisting essentially of urea in the form of urea particles having a size greater than 15 SGN to less than 200 SGN, e.g., greater than 50 to less than 200 SGN, e.g., greater than 80 SGN to less than 200 SGN, e.g., greater than 100 SGN to less than 200 SGN, e.g., greater than 130 SGN to less than 200 SGN, e.g., about 135 SGN, 145 SGN, 150 SGN, 155 SGN, 160 SGN, 165 SGN, 170 SGN, 175 SGN, 180 SGN, 185 SGN, 190 SGN, 195 SGN, 196 SGN, 197 SGN, 198 SGN, or 199 SGN. In another embodiment, provided is a composition consisting essentially of urea in the form of urea particles having a size greater than 15 SGN to less than 200 SGN, wherein about 50% of the urea particles have the described particle size, and more typically about, e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79,%, 80%, 81%, 82%, 83%, 84%, 85%, 86%, 87%, 88%, 89%, 90%, 91%, 92%, 93%, 94%, 95%, 96%, 97%, 98%, 99% or 100% have the described particle size.

In another embodiment, the composition having appropriately sized urea particles further comprise other ingredients selected from the group consisting of proteins, vitamins, minerals, carbohydrates, phosphorous based compounds, potassium based compounds, and/or a combination thereof. For example, a composition having appropriately sized urea particles and a combination of proteins, vitamins, minerals, carbohydrates, phosphorous based compounds, and potassium based compounds. The composition may be in a form selected from the group consisting of a pellet, meal, liquid, suspension, block, solution and spray. In some embodiments, the composition is in a pellet form.

In another embodiment, the composition comprises urea particles of appropriate size and other ingredients, wherein the urea particles and other ingredients form a homogeneous mixture. In one embodiment, the composition comprising urea particles and other ingredients as described herein to form a mixture that is free flowing in character.

In another embodiment, a method of making urea particles having a size greater than about 15 SGN to less than about 200 SGN is provided, e.g., greater than about 50 to less than about 200 SGN, e.g., greater than about 80 SGN to less than about 200 SGN, e.g., greater than about 100 SGN to less than about 200 SGN, e.g., greater than about 130 SGN to less than about 200 SGN, e.g., about 135 SGN, 145 SGN, 150 SGN, 155 SGN, 160 SGN, 165 SGN, 170 SGN, 175 SGN, 180 SGN, 185 SGN, 190 SGN, 195 SGN, 196 SGN, 197 SGN, 198 SGN, or 199 SGN. In another embodiment, a method of making urea particles having a size greater than about 15 SGN to less than about 200 SGN is provided, wherein about 50% of the urea particles have the described particle size, and more typically about, e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79,%, 80%, 81%, 82%, 83%, 84%, 85%, 86%, 87%, 88%, 89%, 90%, 91%, 92%, 93%, 94%, 95%, 96%, 97%, 98%, 99% or 100% have the described particle size.

In another embodiment, a method of making urea particles having a size greater than 15 SGN to less than 200 SGN is provided, e.g., greater than 50 to less than 200 SGN, e.g., greater than 80 SGN to less than 200 SGN, e.g., greater than 100 SGN to less than 200 SGN, e.g., greater than 130 SGN to less than 200 SGN is provided, e.g., about 135 SGN, 145 SGN, 150 SGN, 155 SGN, 160 SGN, 165 SGN, 170 SGN, 175 SGN, 180 SGN, 185 SGN, 190 SGN, 195 SGN, 196 SGN, 197 SGN, 198 SGN, or 199 SGN. In another embodiment, a method of making urea particles having a size greater than 15 SGN to less than 200 SGN is provided, wherein about 50% of the urea particles have the described particle size, and more typically about, e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79,%, 80%, 81%, 82%, 83%, 84%, 85%, 86%, 87%, 88%, 89%, 90%, 91%, 92%, 93%, 94%, 95%, 96%, 97%, 98%, 99% or 100% have the described particle size.

The method as described herein produces urea particles that can be used as an ingredient or nutrient in animal feed, plant fertilizer, resin production, ethanol production, yeast fermentation and cellulose paper manufacturing.

In an embodiment, the method of making urea particles comprises the steps of: (a) providing urea particles having a size greater than about 200 SGN, e.g. greater than about 200 SGN to less than about 450 SGN, e.g., 200 SGN, 250 SGN, 300 SGN, 350 SGN, 400 SGN, 450 SGN; (b) optionally conditioning the urea particles provided in step (a); (c) cracking the urea particles having a size greater than about 200 SGN to obtain urea particles having a size greater than about 15 SGN to less than about 200 SGN, e.g., greater than about 50 to less than about 200 SGN, e.g., greater than about 80 SGN to less than about 200 SGN, e.g., greater than about 100 SGN to less than about 200 SGN, e.g., greater than about 130 SGN to less than about 200 SGN, e.g., about 135 SGN, 145 SGN, 150 SGN, 155 SGN, 160 SGN, 165 SGN, 170 SGN, 175 SGN, 180 SGN, 185 SGN, 190 SGN, 195 SGN, 196 SGN, 197 SGN, 198 SGN, or 199 SGN.

Note that “conditioning” as used herein in step (b) refers to loosening and pulling apart the urea particles provided in step (a). Conditioning, if performed, does not crack any provided urea particles in step (a) but only separates urea particles that stick together to form a rigid mass.

In another embodiment, the urea particles obtained in step (c) have a size greater than about 15 SGN to less than about 200 SGN, wherein about 50% of the urea particles have the described particle size, and more typically about, e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79,%, 80%, 81%, 82%, 83%, 84%, 85%, 86%, 87%, 88%, 89%, 90%, 91%, 92%, 93%, 94%, 95%, 96%, 97%, 98%, 99% or 100% have the described particle size. In another embodiment, the urea particles provided in step (a) have a size greater than 200 SGN, e.g. greater than about 200 SGN to less than about 450 SGN, e.g., 200 SGN, 250 SGN, 300 SGN, 350 SGN, 400 SGN, 450 SGN, wherein about 50% of the urea particles have the described particle size, and more typically about, e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79,%, 80%, 81%, 82%, 83%, 84%, 85%, 86%, 87%, 88%, 89%, 90%, 91%, 92%, 93%, 94%, 95%, 96%, 97%, 98%, 99% or 100% have the described particle size.

In another embodiment, the urea particles provided in step (a) have a size greater than 200 SGN. In an embodiment, the urea particles provided in step (a) have a size greater than about 200 SGN to less than 450 SGN. In an embodiment, the urea particles obtained in step (c) have a size greater than 15 SGN to less than 200 SGN, e.g., greater than 50 to less than 200 SGN, e.g., greater than 80 SGN to less than 200 SGN, e.g., greater than 100 SGN to less than 200 SGN, e.g., greater than 130 SGN to less than 200 SGN, e.g., about 135 SGN, 145 SGN, 150 SGN, 155 SGN, 160 SGN, 165 SGN, 170 SGN, 175 SGN, 180 SGN, 185 SGN, 190 SGN, 195 SGN, 196 SGN, 197 SGN, 198 SGN, or 199 SGN. In another embodiment, the urea particles obtained in step (c) have a size greater than 15 SGN to less than 200 SGN, wherein about 50% of the urea particles have the described particle size, and more typically about, e.g., 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79,%, 80%, 81%, 82%, 83%, 84%, 85%, 86%, 87%, 88%, 89%, 90%, 91%, 92%, 93%, 94%, 95%, 96%, 97%, 98%, 99% or 100% have the described particle size. In an embodiment, the urea particles in step (a) of the methods are in prill form. In another embodiment, the urea particles in step (a) of the methods are in granular form.

In one embodiment, the urea particles obtained in step (c) are substantially free flowing in character.

In one embodiment, the urea particles provided in step (a) are coated, for example, with formaldehyde, sulfur, polymer (e.g., slow release polymers), fat (e.g., soy fat) or a combination thereof. Coated urea particles are less hygroscopic, and therefore, exhibit better storage stability. In another embodiment, a percentage of the urea particles provided in step (a) are coated as described herein, e.g., greater than 0%, 1%, 2%, 3%, 4%, 5%, 6%, 7%, 8%, 9%, 10%, 25%, 50%, 75% or substantially all of the urea particles in step (a) are coated. In another embodiment, the urea particles provided in step (a) are partially coated, i.e., at least some portion of the urea particle provided in step (a) is coated while some portion of the urea particle in step (a) is uncoated. In another embodiment, a percentage of the urea particles provided in step (a) are partially coated, e.g., greater than 0%, 1%, 2%, 3%, 4%, 5%, 6%, 7%, 8%, 9%, 10%, 25%, 50%, 75% or substantially all of the urea particles in step (a) are partially coated. In another embodiment, some of the urea particles provided in step (a) are coated and partially coated, e.g., greater than 0%, 1%, 2%, 3%, 4%, 5%, 6%, 7%, 8%, 9%, 10%, 25%, 50%, 75% or substantially all of the urea particles in step (a) are coated and partially coated. In another embodiment, the urea particles provided in step (a) consists of coated and partially coated urea particles. In another embodiment, the urea particles provided in step (a) are uncoated, coated, partially coated, or a combination thereof.

In one embodiment, the method of making urea particles as described herein further comprises step (d) optionally coating the urea particles obtained in step (c) with a coating material, for example, formaldehyde, sulfur, polymer, fat or a combination thereof. In another embodiment, the method of making urea particles as described herein further comprises partially coating the urea particles obtained in step (c) with a coating material, for example, formaldehyde, sulfur, polymer, fat or a combination thereof.

Embodiments in accordance with the present disclosure provide urea particles having a substantially uniform range of SGN sizes (15-200) and exhibit increased surface area based on the cracking process. As described in more detail below, utilization of this uniformly ranged size of urea particles, having increased surface area, provides a surprising advantage over conventional urea (large size). The advantages are particularly apparent in that it is more effectively combined with other ingredients and its usage is more energy efficient in developing urea based formulations.

In more detail, the appropriately sized urea particles obtained according to the methods described herein allow for efficient processibility of the urea particles in various applications. For instance, the urea particles obtained according to the methods described herein are sized to provide more total surface area compared to conventional urea, for example, urea provided in step (a). Without being bound by any theory, the urea particles obtained from the methods described herein (after step (c)) melt faster relative to urea particles provided in step (a) potentially because of the increased surface area of the urea particles obtained from the disclosed methods. For example, in embodiments where the urea particles provided in step (a) are coated or partially coated, the urea particles obtained by the methods disclosed herein melt faster relative to urea particles provided in step (a) potentially due to increased surface area and severance in coating of the obtained urea particles. The faster melting of the urea particles is helpful, for example, in processing or manufacturing formulations, including but not limited to solid or liquid formulations. Additionally, the faster melting of urea particles are also helpful in energy efficient manufacturing of various formulations. For example, liquid formulation manufacturing using urea is an endothermic process requiring external energy input. The urea particles described herein melt faster during liquid formulation manufacturing thus requiring less energy input compared to urea having bigger size than the urea particles obtained by methods disclosed herein.

In some instances, the urea particles obtained from the methods described herein have appropriate size for animal feed pellet manufacturing. The feed pellets manufactured using large size urea particles may break apart, thereby, decreasing stability of the feed pellets. Additionally, the large sized urea particles have a tendency to protrude out of the feed pellets making these feed pellets unstable and prone to fragmentation, making the pellets less desirable for animal consumption. The urea particles obtained from the methods described herein overcome both of the stability and animal consumption issues.

In other instances, the urea particles obtained from the methods disclosed herein assimilate efficiently with other ingredients in animal feed and plant fertilizer, for example, urea with more surface area more efficiently and quickly combine with other ingredients or nutrients.

The urea particles obtained from the methods disclosed herein also aid in efficient fermentation and reduced ferment time for resin production, ethanol production, yeast fermentation, and cellulose paper manufacturing. The urea particles obtained according to the methods described herein allow for inclusion of higher quantity of urea particles in the animal feed, such as in producing homogeneous mixture or feed pellets, thereby, improving the urea bioavailability or animal palatability.

The urea particles obtained from the methods described herein, can be used without further coating or in the alternative, be coated, or partially coated before further use. The increased surface area of the urea particles obtained according to the disclosed methods, allows for more efficient coating, thereby, improving storage stability of urea particles alone or in combination with other ingredients. Such efficient coating facilitates slow release of nitrogen such as in plant fertilizer, thereby, enhancing bioavailability.

In one embodiment, the urea particles obtained according to the methods disclosed herein, can be further combined with other ingredients for example a protein, a vitamin, a mineral, a carbohydrate, a phosphorous based compound, a potassium based compound, and a combination thereof.

In one embodiment, the urea particles obtained by the methods described herein are free flowing in character.

In one embodiment, the urea particles obtained by the methods described herein have a desired size so that the combination with the other ingredients produces a homogeneous mixture of urea particles and other ingredients.

The urea particles disclosed could be used, but not limited to, in plant nutrition, animal nutrition, resin production, ethanol production, yeast fermentation and cellulose paper manufacturing.

The words “comprise”, “comprising”, “include” and “including” when used in this specification and in the following claims are intended to specify the presence of the stated features, integers, components, or steps, but they do not preclude the presence or addition of one or more additional features, integers, components, or steps thereof.

The term Size Guide Number (SGN) as used herein is the calculated diameter of the “average particle” expressed in millimeters to the second decimal and then multiplied by 100. For example, a 5 mm size particle has a SGN number of 500.

While the methods disclosed herein have been described and shown with reference to particular operations performed in a particular order, it will be understood that these operations may be combined, sub-divided, or re-ordered to form equivalent methods without departing from the teachings of the present invention. Accordingly, unless specifically indicated herein, the order and grouping of the operations are not a limitation of the present invention.

It should be appreciated that reference throughout this specification to “one embodiment” or “an embodiment” or “one example” or “an example” means that a particular feature or characteristic described in connection with the embodiment may be included, if desired, in at least one embodiment of the present invention. Therefore, it should be appreciated that two or more references to “an embodiment” or “one embodiment” or “an alternative embodiment” or “one example” or “an example” or “another embodiment” in various portions of this specification are not necessarily all referring to the same embodiment.

It should be appreciated that in the foregoing description of exemplary embodiments of the invention, various features of the invention are sometimes grouped together in a single embodiment, or description thereof for the purpose of streamlining the disclosure and aiding in the understanding of one or more of the various inventive aspects. Inventive aspects lie in less than all features of a single foregoing disclosed embodiment, and each embodiment described herein may contain more than one inventive feature.

While the invention has been particularly shown and described with reference to embodiments thereof, it will be understood by those skilled in the art that various other changes in the form and details may be made without departing from the spirit and scope of the invention.

### EXAMPLES

The following example is provided for illustrative purposes only and is not intended to limit the scope of the invention.

### Example 1

A batch of 100 kg urea having a particle size of about 280 SGN size was cracked for about 15 to 30 seconds to obtain urea particles having a size less than 200 SGN wherein at least 50% of the cracked urea particles have a size less than 200 SGN.

What is claimed is:

1. A composition comprising urea wherein the urea is in the form of urea particles having a size from greater than about 15 SGN to less than about 200 SGN.

2. The composition of claim 1, wherein the urea particles have a size from greater than about 80 to less than about 200 SGN.

3. The composition of claim 1, wherein the urea particles have a size from greater than about 130 to less than about 200 SGN.

4. The composition of claim 1, wherein at least 50% of urea particles have the described size.

5. A method of making urea particles comprising the steps of:
(a) providing urea particles having a size greater than about 200 SGN;
(b) optionally conditioning the urea particles provided in step (a);
(c) cracking the urea particles having a size greater than about 200 SGN to obtain urea particles having a size from greater than about 15 SGN to less than about 200 SGN.

6. The method of claim 5, wherein the urea particles in step (a) have size from greater than about 200 SGN to less than about 450 SGN.

7. The method of claim 5, wherein the urea particles in step (a) have a size greater than 200 SGN.

8. The method of claim 5, wherein the urea particles in step (c) have a size from greater than 15 SGN to less than 200 SGN.

9. The method of claim 5, wherein at least 50% of the urea particles provided in step (a) have the described size.

10. The method of claim 5, wherein at least 50% of the urea particles obtained in step (c) have the described size.

11. The composition of claim 1, wherein the urea particles are used in animal feed.

12. The composition of claim 1, wherein the urea particles are used in plant fertilizer.

13. The method of claim 4, further comprising (d) formulating the urea particles from step (c) into an animal feed.

14. The method of claim 5, further comprising (d) formulating the urea particles obtained from step (c) into a plant fertilizer.

15. The method of claim 5, wherein the urea particles in step (a) are coated.

16. The method of claim 5, further comprising (d) coating the urea particles obtained in step (c).

17. The method of claim 5, wherein the urea particles obtained in step (c) are in substantially free-flowing form.

18. An animal feed comprising a homogenous mixture of urea particles and another animal feed ingredient, wherein the animal feed is free flowing and at least 50% of the urea particles have a size great than 15 SGN and less than 200 SGN.

19. The animal feed of claim 18, wherein the animal ingredient is a protein.

20. The animal feed of claim 19, wherein the urea particles are coated with a fat.
